(19) United States
(12) Patent Application Publication
(10) Pub. No.: US 2014/0154289 A1
(43) Pub. Date: Jun. 5, 2014

(54) ATTENUATED PLASMODIUM WITH DEACTIVATED HMGB2 GENE, AS VACCINE

(71) Applicants: Catherine Vaquero, Paris (FR); Sylvie Briquet, Ecouen (FR); Nadou Essenia Lawson-Hogban, Serris (FR); Salaheddine Mecheri, Fontenay Le Fleury (FR); Robert Menard, Clamart (FR)

(72) Inventors: Catherine Vaquero, Paris (FR); Sylvie Briquet, Ecouen (FR); Nadou Essenia Lawson-Hogban, Serris (FR); Salaheddine Mecheri, Fontenay Le Fleury (FR); Robert Menard, Clamart (FR)

(73) Assignees: INSTITUT PASTEUR, Paris Cedex 15 (FR); UNIVERSITE PIERRE ET MARIE CURIE (PARIS 6), PARIS (FR)

(21) Appl. No.: 14/233,632

(22) PCT Filed: Jul. 19, 2012
(86) PCT No.: PCT/FR2012/051722

§ 371 Date: Jan. 17, 2014

(30) Foreign Application Priority Data
Jul. 20, 2011 (FR) 1156571

(51) Int. Cl. A61K 39/015

(52) U.S. Cl. CPC A61K 39/015
USPC 424/272.1

(57) ABSTRACT

The present invention relates to novel compositions and methods for immunizing a host against malaria using a Plasmodium parasite genetically attenuated via the inactivation of the function of the hmgb2 gene.

### CROSS-REFERENCE TO RELATED APPLICATIONS

This application is the U.S. National Stage under 37 U.S.C. §371 of International Application No. PCT/FR2012/051722, filed Jul. 19, 2012, which claims priority to French Application No. 1156571, filed Jul. 20, 2011. The International Application published on Jan. 24, 2013 as WO 2013/011247. All of the above applications are incorporated by reference in their entireties.

### SEQUENCE LISTING

The instant application contains a Sequence Listing which has been submitted in ASCII format via EFS-Web and is hereby incorporated by reference in its entirety. Said ASCII copy, created on Jan. 15, 2014, is named B1171PC-Seq_listing_ST25, and is 10,172 bytes in size.

### FIELD OF THE INVENTION

The present invention falls within the field of medicine, and more particularly that of combating malaria.

### TECHNOLOGICAL BACKGROUND OF THE INVENTION

Malaria is an infectious disease caused by a eukaryotic single-cell parasite of the Plasmodium genus. This parasitic disease is present throughout the world and causes serious economic and health problems in developing countries. P. falciparum is the most harmful species of the five types of Plasmodium infecting humans. According to the World Health Organization (WHO), P. falciparum is responsible for 250 to 500 million cases of acute disease and approximately one million deaths each year (especially children less than 5 years old and pregnant women). Cerebral malaria is a severe neurological complication of malaria which is responsible for the vast majority of lethal cases of the disease. Even if the individual survives, cerebral malaria can lead to serious neurological after effects, in particular in young children, whose immune system is in the process of forming. The pathogenesis of cerebral malaria is complex and still far from being completely elucidated. At the current time, it is accepted that the cerebral pathology is probably the result of the sequestration of parasitized red blood cells in the microvessels of the main organs (spleen, lungs, heart, intestines, kidneys, liver and brain) and of the production of pro-inflammatory cytokines in these same organs, resulting in a systemic syndrome and state which can lead to the death of the individual.

Combating malaria is one of the major challenges for the WHO but, to date, all efforts aimed at controlling this disease have failed. During the past thirty years, even though WHO figures appear to be encouraging, the situation has worsened because of the occurrence of the resistance of anopheles mosquitoes to insecticides and of the growing chemoresistance of P. falciparum to antimalarial drugs (even used in combinations).

Combating malaria is made difficult by the absence of a vaccine which is actually effective against the disease. The first attempts at developing a vaccine go back to the 1970s. Since then, there has been an increasing number of vaccine trials, but the latter are faced with the complexity of the development of the parasite in its two successive hosts, humans and mosquitoes, and also with an extremely complicated mechanism for evading the immune system involving a considerable antigenic variation of the parasite.

This variation during the erythrocytic phase of the parasite makes conventional preventive vaccination using Plasmodium protein-peptide complexes extremely difficult. A malaria vaccine produced from proteins of the parasite is currently in phase III of clinical trials (RTS,S from GlaxoSmithKline Biologicals). However, the results obtained during phase II show that this vaccine reduces the occurrence of clinical malaria by only 35% and that of severe malaria by only 49%.

Obtaining a vaccine which is effective against the erythrocytic forms of the parasite is nevertheless a major challenge in the context of eradication of the disease, given that such a vaccine would make it possible both to reduce the symptoms and also the parasite load and the amount of gametocytes in the blood and therefore to reduce transmission of the parasite.

A recent study has shown that the repeated administration of very low doses of red blood cells parasitized by P. falciparum 3D7 combined with the simultaneous taking of antimalarial drugs induces an immunity capable of protecting patients against the erythrocytic phase of a homologous strain (Pombo et al., 2002).

It has therefore been envisaged to use genetically attenuated live parasites (or GAPs, “Genetically Attenuated Parasites”) to induce long-lasting sterile protection. Several GAPs have thus been described. However, the majority of these GAPs target the hepatocyte stage via the sporozoites of the attenuated parasites. Two GAPs which target the erythrocytic stage have also been described: P. yoelii rodent parasites in which the purine nucleoside phosphorylase (PNP) (Ting et al., 2008) or the nucleoside transporter 1 (NT1) (Aly et al., 2010) has been inactivated. It has been shown that these GAPs confer immunity with respect to a homologous strain, but also, to a lesser extent, with respect to heterologous strains. The efficacy and the innocuousness of the vaccines using these genetically attenuated live parasites remain, however, to be determined.

At this time, it therefore remains essential to develop novel strategies which would make it possible in particular to curb the occurrence of serious attacks such as cerebral malaria, but also to reduce the parasite load and the amount of gametocytes in peripheral blood and thus to reduce the risks of transmission of the disease.

### SUMMARY OF THE INVENTION

The objective of the present invention is to provide novel vaccine compositions for preventing malaria, and more particularly the occurrence of serious attacks of malaria such as cerebral malaria.

The inventors have demonstrated that a live parasite belonging to the Plasmodium genus and in which the function of the hmgb2 gene has been inactivated can be used as an immunogen in a malaria vaccine. They have in fact observed that the administration of such a parasite induces an immune reaction in the host which makes it possible to obtain both clearance of the parasite (parasite load below the threshold of detection by microscopy in peripheral blood) and also effective and long-lasting protection against an infection with a Plasmodium, in particular with a highly pathogenic strain, and more particularly against the erythrocytic phase of the parasite which is responsible for the symptomatology of the disease and for its transmission.

Thus, the present invention relates first of all to a live parasite belonging to the Plasmodium genus in which the function of the hmgb2 gene is inactivated, for use in the prevention of malaria, and more particularly in the prevention of cerebral malaria which is a severe neurological complication of the disease.

Preferably, the parasite according to the invention is used in the prevention of malaria or cerebral malaria in a mammal, in particular a human being.

The strain of the parasite can be selected from the group consisting of Plasmodium berghei, Plasmodium falciparum, Plasmodium vivax, Plasmodium ovale, Plasmodium malariae and Plasmodium knowlesi. Preferably, the strain of the parasite is selected from the group consisting of Plasmodium falciparum, Plasmodium vivax, Plasmodium ovale, Plasmodium malariae and Plasmodium knowlesi, more particularly preferably from the group consisting of Plasmodium falciparum, Plasmodium vivax, Plasmodium ovale and Plasmodium malariae.

According to one particular embodiment, the strain of the parasite is Plasmodium falciparum.

According to another particular embodiment, the strain of the parasite is Plasmodium berghei, in particular Plasmodium berghei NK65.

Preferably, the parasite in its wild-type form does not cause cerebral malaria.

According to one particular embodiment, the strain of the parasite is a Plasmodium falciparum strain which is non-cytoadherent or which has a reduced cytoadherence capacity, preferably which has a reduced cytoadherence capacity.

The parasite according to the invention may be in an intra-erythrocytic form, in particular in the form of intra-erythrocytic trophozoites, merozoites or schizonts, preferably in the form of intra-erythrocytic merozoites or schizonts.

The parasite according to the invention may be in the form of non-intra-erythrocytic merozoites, i.e. of merozoites obtained from parasitized red blood cells by total or partial purification.

The parasite according to the invention may also be in the form of sporozoites.

The function of the hmgb2 gene can be inactivated by total or partial deletion of said gene, preferably by total deletion of said gene. In particular, the coding region of the hmgb2 gene can be replaced with a selectable marker, such as the human gene encoding dihydrofolate reductase.

The function of the hmgb2 gene can also be inactivated by means of an interfering RNA which blocks or decreases the translation of the HMGB2 protein.

In the parasite according to the invention, the function of one or more other genes can be inactivated. In particular, the other gene(s) of which the function is inactivated can be chosen from the group consisting of the genes encoding purine nucleoside phosphorylase, nucleoside transporter 1, UIS3, UIS4, p52 and p36, and a combination thereof.

In a second aspect, the present invention relates to an immunogenic composition comprising, as immunogen, an immunologically effective amount of a parasite according to the invention, and one or more pharmaceutically acceptable excipients or supports. The present invention also relates to a malaria vaccine comprising, as immunogen, a parasite according to the invention, and one or more pharmaceutically acceptable excipients or supports.

The immunogenic composition or the vaccine may also comprise one or more immunological adjuvants. In particular, the immunological adjuvant(s) may be selected from the group consisting of adjuvants of muramyl peptide type; trehalose dimycolate (TDM); lipopolysaccharide (LPS); monophosphoryl lipid A (MPL); carboxymethylcellulose; complete Freund's adjuvant; incomplete Freund's adjuvant; adjuvants of “oil-in-water” emulsion type, optionally supplemented with squalene or squalane; mineral adjuvants; bacterial toxins; CpG oligodeoxynucleotides; saponins; synthetic copolymers; cytokines and imidazoquinolones.

The immunogenic composition or the vaccine may be formulated so as to be administered parenterally.

The immunogenic composition or the vaccine may comprise several parasites according to the invention.

Preferably, the composition or the vaccine comprises between 10 and 10⁷, preferably between 10 and 10⁵, and more particularly preferably between 10² and 10⁵ parasites per injection dose.

According to one particular embodiment, the immunogenic composition or the vaccine comprises between 10 and 10⁶, preferably between 10² and 10⁶, parasites according to the invention in an intra-erythrocytic form per dose unit.

According to another particular embodiment, the immunogenic composition or the vaccine comprises between 10² and 10⁷ parasites according to the invention in the form of non-intra-erythrocytic merozoites per dose unit.

According to yet another particular embodiment, the immunogenic composition or the vaccine comprises between 10³ and 10⁷ parasites according to the invention in the form of sporozoites per dose unit.

In another aspect, the present invention relates to the use of a parasite according to the invention, for preparing a vaccine composition against malaria or cerebral malaria.

In another aspect, the present invention also relates to a method for immunizing a subject against malaria, comprising the administration of an immunogenic composition or of a vaccine according to the invention to said subject, preferably parenterally, in particular subcutaneously, intramuscularly, intradermally or intravenously.

### BRIEF DESCRIPTION OF THE DRAWINGS

FIG. 1: Parasitaemia of C57BL/6 mice infected with red blood cells parasitized (pRBCs) with the wild-type parasite PbNK65 or the mutated parasite PbNK65 Δhmgb2 (10⁵ and 10⁶ pRBC per intravenous injection, respectively). The parasitaemia is analysed by counting pRBCs in stained smears under a microscope and is presented as the percentage of pRBCs (mean±standard deviation). The experiment was carried out with 5 mice and repeated several times. The p value is 0.008.

FIG. 2: Study of the survival of mice subjected to a primary infection with the mutated parasite PbNK65 Δhmgb2 and subsequently infected three times with the two pathogenic wild-type parasites PbNK65 and PbANKA. The first challenge was carried out on D19 (19 days after the primary infection), the second on D71 and, finally, the third on D162. At each challenge, the pathogenicity of the two preparations of pRBCs infected with PbNK65 and PbANKA was verified by infecting mice of the same age which had not been subjected to primary infection and by monitoring what became of them and their death due to hyperparasitaemia or cerebral malaria. Five mice were analysed for each of the experiments.

FIG. 3: Immunization of C57BL/6 mice with various amounts of pRBCs infected with the mutated parasite PbNK65 Δhmgb2. Ten mice were infected at time 0 with 10³ (□), 10⁴ (∘) or 10⁵ (Δ) pRBCs infected with PbNK65 Δhmgb2. These mice were then subjected to an infection challenge on D24 with 10⁵ pRBCs infected with the wild-type pathogenic parasite PbNK65 (n=5) or PbANKA (n=5). Naive mice were also subjected to an infection challenge on D24 with 10⁵ pRBCs infected with the wild-type pathogenic parasite PbNK65 (▴) or PbANKA (•).

### DETAILED DESCRIPTION OF THE INVENTION

The sequencing of the P. falciparum genome was completed in 2002. This genome comprises approximately 5500 genes, more than 50% of which have a completely unknown function. When studying the factors involved in transcriptional regulation in P. falciparum, the inventors annotated four HMGBs (PfHMGB1 to 4; High Mobility Group Box proteins). These are very abundant non-histone nuclear proteins which have the particularity of being both nuclear factors and extracellular mediators.

In humans, where they have been particularly studied, they are present in the cell nucleus and act as architectural factors, participating in the regulation of transcription via chromatin remodelling. In situations of danger for the cell (foreign body, inflammation), these proteins are actively secreted into the cell medium and/or passively secreted in the case of cell necrosis, and act as actual danger signals (alarmins) by activating macrophages and other competent cells of the immune system (Lotze and Tracey, 2005). The involvement of the human HMGB1 protein has been demonstrated in diseases which cause a systemic inflammation, such as lupus erythematosus, septic shock or rheumatoid arthritis, and in certain cancers. Finally, the increase in the human HMGB1 protein, in the sera of patients, has been correlated with the severity of these diseases.

In P. falciparum, the inventors have more particularly studied PfHMGB1 and PfHMGB2, which are small proteins of less than 100 amino acids. These two proteins, like their human homologues, are capable of bending DNA and interacting with particular DNA structures (Briquet et al., 2006). They are also secreted and found in P. falciparum culture supernatants. The HMGB1 and 2 proteins of P. falciparum are capable of activating and inducing TNFα expression in mouse monocyte lines (Kumar et al., 2008).

In the present application, the inventors have used, as an animal model of cerebral malaria, C57BL/6 mice infected with the P. berghei ANKA parasite (PbANKA). This animal model reproduces the main characteristics of the human pathology, such as ataxia, spatial disorientation, convulsions and coma. These clinical symptoms can result in death of the animal. As in humans, the sequestration of parasitized red blood cells, cerebral haemorrhages and lesions, the production of pro-inflammatory cytokines and the destruction of the blood-brain barrier are observed in this model. In this model, all of the C57BL/6 mice infected with the PbANKA isolate die from cerebral malaria in approximately 7 days. On the other hand, the inventors have observed that 60% of mice infected with the parasite PbANKA Δhmgb2 do not develop cerebral malaria.

The inventors have inactivated the hmgb2 gene of another P. berghei isolate, the NK65 isolate, which induces the death of C57BL/6 mice due to hyperparasitaemia in 20 to 25 days after infection, but does not cause cerebral malaria. They have first of all observed that the inactivation of this gene hinders the development of PbNK65 Δhmgb2 in the mouse with clearance of the parasite from peripheral blood obtained 10 to 15 days after infection. The inventors have subsequently demonstrated that the host's response resulting in clearance of the parasite is sufficient to induce sterile immunity lasting more than six months, not only with respect to the homologous wild-type strain PbNK65, but also with respect to the highly pathogenic heterologous strain PbANKA which is capable of inducing cerebral malaria.

Thus, in a first aspect, the present invention relates to a live parasite belonging to the Plasmodium genus in which the function of the hmgb2 gene is inactivated, for use in the prevention of malaria or of cerebral malaria, preferably in a mammal, and quite particularly preferably in a human being.

The parasite is preferably capable of developing in vertebrates, and more particularly in mammals, and belongs to the subgenus selected from the group consisting of Plasmodium vinckeia, Plasmodium plasmodium and Plasmodium Laverania.

According to one embodiment, the parasite is capable of developing in a human host and belongs to the subgenus Plasmodium plasmodium or Plasmodium Laverania. Preferably, the parasite belongs to a species responsible for malaria in humans, more particularly to a species selected from the group consisting of Plasmodium falciparum, Plasmodium vivax, Plasmodium ovale, Plasmodium malariae and Plasmodium knowlesi. Although Plasmodium knowlesi is a parasite which infects essentially primates, an increasing number of cases of human infections with this parasite are being reported. More particularly preferably, the parasite belongs to a species selected from the group consisting of Plasmodium falciparum, Plasmodium vivax, Plasmodium ovaleand Plasmodium malariae. According to one particular embodiment, the parasite belongs to a species selected from the group consisting of Plasmodium falciparum, Plasmodium vivax and Plasmodium malariae. According to one preferred embodiment, the parasite belongs to the species Plasmodium falciparum.

According to another embodiment, the parasite belongs to a species which is capable of inducing an immune reaction but is not capable of causing the symptoms of malaria in human beings. Preferably, this parasite is a rodent parasite belonging to the subgenus Plasmodium vinckeia. The use of rodent parasites in the context of vaccination in humans makes it possible to considerably reduce the risks associated with the administration of live parasites to the subject. The rodent parasite can be modified so as to express one or more proteins of a Plasmodium which infects humans, such as P. falciparum, which is or are required for the invasion of human red blood cells. Such proteins are, for example, described in the article by Triglia et al., 2000. Preferably, the parasite belongs to the species Plasmodium berghei or Plasmodium yoelii. More particularly preferably, the parasite belongs to the species Plasmodium berghei. According to one preferred embodiment, the parasite is the NK65 isolate of the species Plasmodium berghei.

According to one particular embodiment, the parasite belongs to a species selected from the group consisting of Plasmodium berghei, Plasmodium falciparum, Plasmodium vivax, Plasmodium ovale, Plasmodium malariae and Plasmodium knowlesi. The parasite may also belong to a species selected from the group consisting of Plasmodium berghei, Plasmodium falciparum, Plasmodium vivax, Plasmodium ovale and Plasmodium malariae or from the group consisting of Plasmodium berghei, Plasmodium falciparum, Plasmodium vivax and Plasmodium malariae, or else from the group consisting of Plasmodium berghei and Plasmodium falciparum.

According to one preferred embodiment, the wild-type strain of the parasite, i.e. the parasite in which the hmgb2 gene is active, does not cause cerebral malaria. This strain may, for example, be chosen from the group consisting of Plasmodium berghei NK65, Plasmodium vivax, Plasmodium ovale, Plasmodium malariae and Plasmodium knowlesi. This strain may also be a Plasmodium falciparum strain which has lost its cytoadherence capacity or which has a reduced cytoadherence capacity. According to one embodiment, the wild-type strain of the parasite is a non-cytoadherent Plasmodium falciparum strain. According to another embodiment, the wild-type strain of the parasite is a Plasmodium falciparum strain which has a reduced cytoadherence capacity. Cytoadherence is a property of Plasmodium falciparum which is directly linked to the development of cerebral malaria. Indeed, red blood cells infected with a cytoadherent Plasmodium falciparum strain have the capacity to bind to surface molecules of endothelial cells, such as CD36, ICAM1, VCAM1 or PECAM1/CD31, and thus to cause a vascular obstruction, inflammation and damage in various organs, in particular in the brain. The cytoadherence capacity of a strain can be evaluated by any technique known to those skilled in the art, such as, for example, that described in the article by Buffet et al., 1999 or that by Traore et al., 2000. The term “reduced cytoadherence capacity” refers to a cytoadherence capacity which is lower than that observed on a reference cytoadherent Plasmodium strain, for example the Plasmodium falciparum 3D7 strain. The cytoadherence can be reduced by at least 40%, 50%, 60%, 70%, 80%, 90% or 95%, preferably by at least 80%, and more particularly preferably by at least 90%, relative to a reference cytoadherent Plasmodium strain, for example the Plasmodium falciparum 3D7 strain. It is possible to obtain Plasmodium falciparum strains which have a reduced cytoadherence capacity, for example by multiplying the passages in culture ex vivo (Udeinya et al., 1983). Various Plasmodium falciparum strains with a reduced cytoadherence capacity have been described, for example in the article by Trenholme et al., 2000 (Plasmodium falciparum in which the clag9 gene is inactivated) and by Nacer et al., 2011 (Plasmodium falciparum D10 and T9-96). Thus, according to one particular embodiment, the wild-type strain of the parasite is a Plasmodium falciparum strain which is sparingly cytoadherent or non-cytoadherent. In particular, the wild-type strain of the parasite may be a Plasmodium falciparum strain with a reduced cytoadherence capacity, selected from the group consisting of a Plasmodium falciparum strain in which the clag9 gene is inactivated, of the Plasmodium falciparum D10 strain and of the Plasmodium falciparum T9-96 strain.

In the parasite used according to the invention, the function of the hmgb2 gene is inactivated. This means that the activity of the HMGB2 protein encoded by this gene is totally or partially inhibited, preferably totally inhibited. This inhibition can be obtained by numerous methods well known to those skilled in the art. It is thus possible to block the function of the gene at the transcriptional or translational level or at the protein level, for example by blocking or decreasing the transcription or the translation of the hmgb2 gene or by disrupting the correct folding of the protein or its activity.

The function of the hmgb2 gene can in particular be inactivated by the total or partial deletion of this gene, or the insertion or the substitution of one or more nucleotides in order to make this gene inactive.

According to one particular embodiment, the function of the hmgb2 gene is inactivated by total or partial deletion of this gene, preferably by total deletion.

Preferably, the deletion of the hmgb2 gene is obtained by homologous recombination. This method is well known to those skilled in the art and has been applied many times to the parasites of the Plasmodium genus (see, for example, Thathy and Ménard, 2002). According to one particular embodiment, the coding region of the hmgb2 gene is replaced by homologous recombination with a marker which makes it possible to select the parasites in which the recombination has taken place. The selectable marker may be, for example, the human dihydrofolate reductase (dhfr) gene which confers pyrimethamine resistance on the parasite. The obtaining of parasites in which the hmgb2 gene is deleted is exemplified in the experimental section. According to one particular embodiment of the invention, the parasite used is a parasite in which the hmgb2 gene has been replaced with a selectable marker, preferably with the human dhfr gene. According to one very particular embodiment of the invention, the parasite is a Plasmodium berghei, preferably the NK65 isolate, in which the hmgb2 gene has been replaced with a selectable marker, preferably with the human dhfr gene. According to another particular embodiment of the invention, the parasite used is a Plasmodium falciparum, which is preferably non-cytoadherent or sparingly cytoadherent, in which the hmgb2 gene has been replaced with a selectable marker, preferably with the human dhfr gene.

The function of the hmgb2 gene can also be inactivated by blocking or decreasing the translation of the mRNA of this gene. RNA interference, which makes it possible to specifically inhibit the expression of the target gene, is a phenomenon well known to those skilled in the art that has already been used to inhibit the expression of Plasmodium genes (see, for example, McRobert and McConkey, 2002; Mohmmed et al., 2003; Gissot et al. 2004). According to one embodiment, a sequence encoding an interfering RNA, or its precursor, is introduced into the genome of the parasite and its expression is controlled by a strong promoter, preferably a constitutive promoter, such as, for example, the promoter of the eEF 1 a elongation factor, which is active in all stages of the development of the parasite, or the promoter of the HSP70 gene, which is active in the sporozoites and during the erythrocytic cycle. The sequence and the structure of the interfering RNA can be easily chosen by those skilled in the art. In particular, the interfering RNA used may be a small interfering RNA (siRNA).

The GenBank references of the sequences of hmgb2 genes of various Plasmodium species that have been sequenced and also those of the corresponding protein sequences are given in Table 1 below.

TABLE 1
GenBank reference of the nucleotide and protein
sequences of HMGB2 of various Plasmodium species
which have been sequenced to date

| Species | hmgb2 Gene | HMGB2 Protein |
|---|---|---|
| Plasmodium falciparum 3D7 | XM_001349310 | XP_001349346 |
| | (SEQ ID No. 1) | (SEQ ID No. 2) |
| Plasmodium vivax Sal-1 | XM_001614943.1 | XP_001614993.1 |
| | (SEQ ID No. 3) | (SEQ ID No. 4) |
| Plasmodium berghei str. ANKA | XM_667771 | XP_672863 |
| | (SEQ ID No. 5) | (SEQ ID No. 6) |
| Plasmodium knowlesi | XM_002258117 | XP_002258153 |
| | (SEQ ID No. 7) | (SEQ ID No. 8) |
| Plasmodium yoelii | XM_722771 | XP_727864 |
| | (SEQ ID No. 9) | (SEQ ID No. 10) |

The hmgb2 genes of the Plasmodium species that have not yet been sequenced can be easily identified by means of methods well known to those skilled in the art, in particular by hybridization or PCR.

In the parasite according to the invention, the function of one or more genes, other than hmgb2, can also be inactivated. The additional gene of which the function is inactivated can be a gene which participates in the survival of the parasite in a mammalian host, in particular in humans. Preferably, the inactivation of this additional gene makes it possible to attenuate the virulence of the parasite while at the same time preserving its immunogenic nature. This additional gene can be chosen from the group consisting of purine nucleoside phosphorylase (PNP; PFE0660c), nucleoside transporter 1 (NT1; PF13_{—}0252), UIS3 (PF13_{—}0012), UIS4 (PF10_{—}0164 early transcript), p52

(PFD0215c protein with 6-cysteine motif) and p36 (PFD0210c), and also combinations thereof (the references between parentheses are the PlasmoDB bank accession numbers of the Plasmodium falciparum 3D7 sequences, given here by way of example).

The methods for growing the parasites according to the invention and also the methods of preservation, in particular of cryopreservation, of the parasites have been described previously and are well known to those skilled in the art (see, for example, Leef et al., 1979; Orjih et al., 1980). These parasites can be grown ex vivo using cell cultures or in vivo in an animal, for example a mouse.

According to one embodiment, the parasites according to the invention are used in an erythrocytic form, more particularly in the form of non-intra-erythrocytic merozoites or in the form of intra-erythrocytic merozoites, trophozoites or schizonts.

According to one particular embodiment, the parasites according to the invention are used in the form of intra-erythrocytic merozoites, trophozoites or schizonts, i.e. which are inside red blood cells.

According to another particular embodiment, the parasites are used in the form of non-intra-erythrocytic merozoites, i.e. of merozoites which have been partially or totally purified after rupturing of parasitized red blood cells. The merozoites can be obtained according to any one of the methods known to those skilled in the art, such as that described in the article by Boyle et al., 2010.

The parasitized red blood cells can be obtained by introduction of the parasite into a host, preferably a human being, and recovery of the red blood cells of the infected host when the parasitaemia reaches a minimum 1%, preferably between 5% and 10%. According to one preferred embodiment, the parasitized red blood cells are recovered from a human host whose blood group is O and who is Rhesus negative.

According to another preferred embodiment, the parasitized red blood cells are obtained by ex vivo infection of human red blood cells, preferably red blood cells which are blood group O and Rhesus negative. Optionally, the parasitized red blood cell cultures can be synchronized so as to obtain predominantly intra-erythrocytic merozoites, trophozoites or schizonts. The methods for ex vivo culturing of Plasmodium parasites are well known to those skilled in the art (see, for example, Trager and Jensen, 1976).

Anticoagulants, such as heparin, can be added to the parasitized red blood cells thus obtained. The parasitized red blood cells can be preserved by freezing in the presence of one or more cryoprotective agents compatible with use in vivo, such as, for example, glycerol or dimethyl sulphoxide (DMSO). The parasitized red blood cells can also be preserved by refrigeration at 4° C. in an appropriate preserving medium, for example SAGM (“Saline Adenine Glucose Mannitol”) medium or a CPD (Citrate Phosphate Dextrose) solution, but for a period not exceeding approximately 45 days.

According to another embodiment, the parasites according to the invention are used in the form of sporozoites. The sporozoites can be obtained by introduction of the parasite into a mosquito host where it will multiply. The sporozoites are then recovered from the salivary glands of the infected mosquitoes. The sporozoites thus obtained can be preserved by freezing, for example in liquid nitrogen, before being thawed in order to be injected live into a host. Alternatively, after recovery from the salivary glands of the mosquitoes, the sporozoites can be preserved by lyophilization or refrigeration before administration.

The administration of the parasite according to the invention to a subject makes it possible, despite a rapid parasite clearance, to induce in the subject an immunity, lasting several months, with respect to an infection with a Plasmodium, in particular a Plasmodium chosen from the group consisting of Plasmodium falciparum, Plasmodium vivax, Plasmodium malariae and Plasmodium knowlesi, preferably Plasmodium falciparum. This immunity can in particular be a cross-immunity with respect to an infection with a Plasmodium strain other than that of the parasite used. In particular, the administration of a parasite according to the invention belonging to a strain which does not cause cerebral malaria can result in a cross-immunity with respect to an infection with a Plasmodium strain capable of causing this severe neurological complication. The parasite according to the invention can therefore be used for the prevention of malaria and/or of cerebral malaria. In particular, the administration of the parasite according to the invention to a subject makes it possible to induce an immunity, lasting several months, with respect to an infection with a Plasmodium falciparum capable of inducing cerebral malaria and thus to prevent malaria and/or cerebral malaria induced by this parasite.

The term “prevention” as used in this document refers to an absence of symptoms or to the presence of reduced symptoms of the disease after contact with the parasite. In particular, the term “prevention of malaria” refers to an absence of symptoms or to the presence of reduced symptoms in the treated subject after contact with a Plasmodium responsible for malaria. The term “prevention of cerebral malaria” refers to an absence of symptoms or to the presence of reduced symptoms in the treated subject after contact with a Plasmodium responsible for malaria and capable of inducing cerebral malaria. This term can also refer to the absence of development of cerebral malaria or to the development of cerebral malaria which is not very severe or which is reduced, in a subject infected with a Plasmodium responsible for malaria and capable of inducing cerebral malaria.

In a second aspect, the present invention relates to an immunogenic composition comprising an immunologically effective amount of a parasite according to the invention, and one or more pharmaceutically acceptable excipients or supports. The parasite included in the composition is as described above.

According to one embodiment, the composition comprises a parasite according to the invention in an erythrocytic form, more particularly in the form of intra-erythrocytic merozoites, trophozoites or schizonts or of non-intra-erythrocytic merozoites, preferably in the form of intra-erythrocytic merozoites, trophozoites or schizonts.

According to one particular embodiment, the composition comprises red blood cells parasitized with the parasite according to the invention and which can be obtained according to the method described above and in the experimental section.

According to another embodiment, the parasite included in the composition is in the form of sporozoites as described above.

The immunogenic composition is capable of inducing, in the subject to whom it is administered, a response of the immune system against the parasite that it contains. The term “immunologically effective amount” as used here refers to an amount of parasites which is sufficient to trigger an immune response in the subject.

Preferably, the immunogenic composition is a malaria vaccine.

According to one embodiment, the composition according to the invention is obtained by suspending parasitized red blood cells, merozoites or sporozoites, preferably parasitized red blood cells or sporozoites, as defined above, in one or more pharmaceutically acceptable excipients. The excipients can be easily chosen by those skilled in the art according to the form of the parasite, intra-erythrocytic, merozoites or sporozoites, and according to the route of administration envisaged. These excipients can in particular be chosen from the group consisting of sterile water, sterile physiological saline and phosphate buffer. Other excipients well known to those skilled in the art can also be used. Preferably, in the case where the composition comprises parasitized red blood cells, the excipient used is an isotonic solution which ensures the integrity of the red blood cells until administration of the composition to the subject. Preferably, the composition also comprises at least one anticoagulant such as heparin.

The composition can also be obtained by mixing parasite sporozoites, as defined above, with a pharmaceutically acceptable support such as, for example, liposomes.

The excipients or supports used are chosen so as to ensure the integrity of the parasitized red blood cells and/or the survival of the sporozoites or of the merozoites. The excipients or supports used are chosen so as to ensure the survival of the parasites of the invention, whatever the form used (merozoites, sporozoites or intra-erythrocytic forms), until the administration of the composition to the subject to be immunized.

According to one preferred embodiment, the subject to be immunized is a mammal, preferably a human being.

The composition according to the invention may be administered, for example, parenterally, cutaneously, mucosally, transmucosally or epidermally. Preferably, the composition is formulated so as to be administered parenterally, in particular subcutaneously, intramuscularly, intravenously or intradermally.

According to one particular embodiment, the parasite is in an erythrocytic form, preferably included in red blood cells, and the composition is formulated so as to be administered parenterally, preferably subcutaneously, intramuscularly, intravenously or intradermally, and quite particularly preferably intravenously.

According to another particular embodiment, the parasite is in the form of sporozoites and the composition is formulated so as to be administered parenterally, preferably subcutaneously, intramuscularly or intradermally, preferably intramuscularly or subcutaneously. The methods for administering compositions comprising live sporozoites are well known to those skilled in the art (see, for example, international patent application WO 2004/045559 and the article by Hoffman et al., 2010).

According to one embodiment, the parasite is in erythrocytic form and included in red blood cells and the composition according to the invention comprises between 10 and 10⁶ parasitized red blood cells (pRBCs) per dose unit, preferably between 100 and 10⁶ pRBCs, particularly preferably between 100 and 10⁵ pRBCs, and quite particularly preferably between 100 and 10⁴ pRBCs per dose unit. According to one particular embodiment, the parasite is in erythrocytic form and included in red blood cells and the composition according to the invention comprises between 10 and 100 parasitized red blood cells (pRBCs) per dose unit.

According to another embodiment, the parasite is in the form of non-intra-erythrocytic merozoites and the composition according to the invention comprises between 10² and 10⁷ merozoites per dose unit, preferably between 10³ and 10⁵ merozoites per dose unit.

According to yet another embodiment, the parasite is in the form of sporozoites and the composition according to the invention comprises between 10³ and 10⁷ sporozoites per dose unit, preferably between 10⁴ and 10⁵ sporozoites per dose unit.

The dose to be administered can be easily determined by those skilled in the art by taking into account the physiological data of the subject to be immunized, such as the age or immune state thereof, the degree of immunity desired, the number of doses administered and the route of administration used. The dose to be administered can also vary according to the parasite preservation mode.

The composition according to the invention may comprise one or more strains of parasites according to the invention. According to one embodiment, the composition comprises at least one Plasmodium falciparum strain and one Plasmodium vivax strain in which the function of the hmgb2 gene is inactivated.

The composition according to the invention may also comprise one or more other genetically attenuated parasites of the Plasmodium genus. These parasites may, for example, exhibit a modification or an inactivation of the function of the purine nucleoside phosphorylase gene, nucleoside transporter 1, UIS3, UIS4, p52 or p36 gene, or be attenuated parasites obtained by irradiation. These parasites preferably belong to a strain selected from the group consisting of Plasmodium falciparum, Plasmodium vivax, Plasmodium ovale, Plasmodium malariae and Plasmodium knowlesi.

The composition according to the invention may also comprise one or more immunological adjuvants. These adjuvants stimulate the immune system and thus reinforce the immune response obtained with respect to the parasite according to the invention. These immunological adjuvants comprise, without being limited thereto, adjuvants of muramyl peptide type, such as N-acetylmuramyl-L-alanyl-D-isoglutamine (MDP) and derivatives thereof; trehalose dimycolate (TDM); lipopolysaccharide (LPS); monophosphoryl lipid A (MPL); carboxymethylcellulose; complete Freund's adjuvant; incomplete Freund's adjuvant; adjuvants of “oil-in-water” emulsion type optionally supplemented with squalene or squalane; mineral adjuvants such as alum, aluminium hydroxide, aluminium phosphate, potassium phosphate or calcium phosphate; bacterial toxins such as cholera toxin subunit B, the inactivated form of pertussis toxin or the thermolabile lymphotoxin from Escherichia coli; CpG oligodeoxynucleotides; saponins; synthetic copolymers such as copolymers of polyoxyethylene (POE) and polyoxypropylene (POP); cytokines; or imidazoquinolones. Combinations of adjuvants may be used. These various types of adjuvants are well known to those skilled in the art. In particular, the composition according to the invention may comprise one or more immunological adjuvants selected from the group consisting of CpG oligodeoxynucleotides and mineral adjuvants, in particular alum, and a combination thereof.

According to another aspect, the invention relates to the use of a live parasite belonging to the Plasmodium genus in which the function of the hmgb2 gene is inactivated, for preparing a vaccine composition against malaria or cerebral malaria.

The invention also relates to a method for producing a vaccine composition against malaria or cerebral malaria according to the invention.

According to one embodiment, the method comprises the step consisting in mixing red blood cells infected with a live parasite according to the invention, with one or more pharmaceutically acceptable excipients or supports. Preferably, the red blood cells are human red blood cells obtained from a host whose blood group is O and who is Rhesus negative.

According to another embodiment, the method comprises the step consisting in mixing live non-intra-erythrocytic merozoites of a parasite according to the invention with one or more pharmaceutically acceptable excipients or supports.

According to yet another embodiment, the method comprises the step consisting in mixing live sporozoites of a parasite according to the invention with one or more pharmaceutically acceptable excipients or supports.

According to one preferred embodiment, the parasites, in the form of parasitized red blood cells, or of merozoites or in the form of sporozoites, are also mixed with one or more immunological adjuvants. These adjuvants may be as defined above.

The method may also comprise a prior step comprising the obtaining of said parasitized red blood cells, of said merozoites or of said sporozoites, for example using the methods described above.

The composition or the vaccine obtained can be preserved before administration, for example frozen or refrigerated if it contains parasitized red blood cells, or frozen, refrigerated or lyophilized if it contains sporozoites or merozoites. Preferably, the composition or the vaccine obtained is preserved frozen before administration. In the case of lyophilization, an appropriate diluent is added to the lyophilisate before administration, for instance sterile water or sterile physiological saline, preferably sterile physiological saline.

The various embodiments concerning the parasite and the composition according to the invention are also envisaged in this aspect.

According to another aspect, the invention relates to a method for immunizing a subject against malaria, comprising the administration of an immunogenic composition or of a vaccine according to the invention to said subject.

Preferably, the subject is a mammal, more particularly a human being.

According to one preferred embodiment, the method comprises the administration of a vaccine according to the invention to said subject.

According to one embodiment, the method comprises the administration of a single dose of the immunogenic composition or of the vaccine according to the invention.

According to another embodiment, the method comprises the administration of a plurality of doses of the immunogenic composition or of the vaccine according to the invention. The immunization of the subject can be obtained after the administration of 2 to 6 doses. Preferably, an interval of approximately 4 to 8 weeks, preferably of approximately 6 to 8 weeks, is observed between each administration.

The inventors have demonstrated that the administration of a single dose comprising 10⁵ red blood cells parasitized with a parasite according to the invention makes it possible to obtain a sterile immunity lasting at least 6 months in a mammal. According to one preferred embodiment, the method comprises the administration of a first dose of the immunogenic composition or of the vaccine according to the invention, followed by the administration of a second dose approximately six months to one year later. Preferably, the doses comprise between 10 and 10⁶, preferably between 100 and 10⁶, parasitized red blood cells, between 10² and 10⁷ merozoites or between 10³ and 10⁷ sporozoites. According to one preferred embodiment, the doses comprise approximately 10³ parasitized red blood cells. According to another preferred embodiment, the doses comprise between 10 and 100 parasitized red blood cells.

The immunity of the subject with respect to malaria or to cerebral malaria may be total or incomplete. In the case of incomplete immunity, the seriousness of the symptoms of the established disease in an immunized subject will be reduced by comparison with those observed in a non-immunized subject. In the case of total immunity, the immunized subject will show no symptom of the disease after a contact with the parasite.

According to one preferred embodiment, the immunity obtained by administering the composition according to the invention is a sterile immunity. This means that the development of the parasites administered is highly modified and that, approximately 2 to 4 weeks after the administration, the parasites are no longer detected in the subject's peripheral blood.

The invention also relates to a method for immunizing a subject against malaria, comprising the administration of a parasite according to the invention in the form of sporozoites to said subject by means of bites by mosquitoes infected with said parasite.

The examples which follow are given for illustrative and non-limiting purposes.

### EXAMPLES

When studying the function of the HMGB proteins, the inventors recently showed that the HMGB2 protein of P. berghei is involved in the establishment of experimental cerebral malaria (ECM) in a model of C57BL/6 mice infected with the P. berghei ANKA parasite (PbANKA). The inactivation of the hmgb2 gene in PbANKA, without hindering the development of the parasite in the C57BL/6 mice, nevertheless induces a delay in the establishment of the ECM, or even complete abolition thereof in 65% of cases. Furthermore, it was shown that the administration of recombinant HMGB2 proteins to mice previously infected with PbANKA Δhmgb2 made it possible to restore the development of cerebral malaria.

### Materials and Methods

### Mice

The mice used are C57BL/6 mice (Charles River Laboratories). These mice are sensitive to experimental cerebral malaria (CM-S).

### Wild-type Parasites

The P. berghei ANKA parasite (PbANKA) (MRA-867) induces the death of C57BL/6 mice due to cerebral malaria in 7 days +/−1. This parasite has a GFP tag under the control of the eef1 promoter (Thaty and Ménard, 2002).

The P. berghei NK65 parasite (PbNK65) (MRA-268) (de Sa et al., 2009) induces the death of C57BL/6 mice due to hyperparasitaemia in 20 to 25 days after infection. On the other hand, this parasite does not cause cerebral malaria.

Obtaining the PbNK65 Δhmgb2 Parasite

### Construction of the Cloning Vector:

The regions which flank the open reading frame in the 5′UTR and 3′UTR positions of the hmgb2 gene, respectively referred to below as HR1 and HR2 (and having a length of approximately 500 bp), were amplified, by PCR from the genomic DNA of the PbNK65 parasite, using specific primers (Table 2) cloned into the pCR®II-TOPO vector (Invitrogen). Escherichia coli One Shot® TOP10 electrocompetent bacteria were then transformed with these constructs and selected on LB medium containing ampicillin and kanamycin.

TABLE 2
÷ Primers for amplifying the HR1 and
HR2 homology regions

| | |
|---|---|
| sense HR1 | 5′CGATGCGGGCCCAAAAAGGTAAATATGAAA |
| | AAGAAAGGTT3′ (SEQ ID No: 11) |
| antisense HR1 | 5′CGATGCCCCGGGTTTGCAATGGAAACATAT |
| | CAGTT3′ (SEQ ID No: 12) |
| sense HR2 | 5′CCAGTGAGTGCGGCCGCGGAGCCTTTTGTA |
| | TGTGCTTTTTG3′ (SEQ ID No: 13) |
| antisense HR2 | 5′AGCTGGCGCGCCAAGTATTGCAGTAGCATT |
| | TTCTTTAAAT3′ (SEQ ID No: 14) |

The primers used for the amplification were designed so as to add, for
HR1: an ApaI restriction site in the 5′ UTR position and a SmaI restriction site in the 3′UTR position, and
HR2: a NotI restriction site in the 5′ UTR position and an AscI restriction site in the 3′UTR position of the sequence. These restriction sites make it possible to insert the PCR fragments into the vector.

The 5′ and 3′ untranslated regions HR1 and HR2 were then inserted into a pBC SK-Hudhfr vector (Stratagene) containing the gene of human dihydrofolate reductase (hudhfr) under the control of the promoter ef1α in the 5′UTR position and of dhfr/ts (dhfr/thymidylate synthase) in the 3′UTR position. HR1 was inserted in the 5′UTR position with respect to the Hudhfr cassette and HR2 in the 3′UTR position. During the homologous recombination, the two HR1 and HR2 regions paired with complementary sequences of the hmgb2 gene in the P. berghei genome, enabling a double homologous recombination at the level of this gene and, as a result, its deletion. The Hudhfr confers pyrimethamine resistance and the mutant parasites were selected using this drug.

### Transfection of Parasites and Selection of Mutant Parasites:

P. berghei merozoites were transfected according to the protocol described by Koning-Ward et al., 2000. All the infections were carried out by intravenous injection.

Two 3-week-old Swiss mice were infected with 2.6×10⁶ PbANKA or PbNK65 pRBCs. When the parasitaemia reached between 2% and 3% (predominant young stages), the blood was taken on heparin by intracardiac puncture (1.5 to 2 ml of blood). The blood was briefly washed in 5 ml of complete culture medium consisting of 4 vol of RPMI 1640 (Invitrogen) supplemented with 1 vol of foetal calf serum (InVitrogen). After centrifugation for 8 min at 450 g, the pRBCs were taken up in 50 ml of complete culture medium. The cell suspension, to which a further 50 ml of complete culture medium were added, was then placed in culture overnight, in a 500 ml Erlenmeyer flask, at 36.5° C., with shaking at 70 rpm and at a pressure of 1.5 to 2 bar.

The following day, after verification by means of smears of the predominant presence of mature schizonts, the pRBC culture was centrifuged for 10 min at 1500 rpm and then the pellet was taken up in 35 ml of complete culture medium in a 50 ml conical tube. Ten millilitres of 50% Nycodenz (Lucron Bioproducts, ½ dilution in 1× PBS) were subsequently very carefully added to the pRBC culture so as to create a density gradient at the bottom of the tube. Twenty minutes of centrifugation at 450 g, AT, without braking, led to the purification of the pRBCs which then form a brown ring in the middle of the tube. The brown ring of pRBCs was collected (˜20 ml) and then washed with 20 ml of complete culture medium. After 8 min of centrifugation at 450 g, the pellet of mature schizonts was finally taken up in 3 ml of complete culture medium which were then dispensed into three 1.5 ml Eppendorf tubes. This operation had to be carried out very delicately since the schizonts are fragiles. At this stage, the amount of schizonts obtained was sufficient to carry out up to 10 independent transfections. After a final spin for 5 sec at 16 000 g, the schizont pellet in each tube was then taken up in 100 μl of electroporation buffer (Nucleofector Solution 88A6, Amaxa) with 5 μg of each of the plasmid constructs obtained (pBC-5′HR1Hudhfr3′HR2 for pbhmgb1 or pbhmgb2), digested beforehand with ApaI and AscI. The parasites were transfected using the U33 programme of the Amaxa Nucleofector electroporator. The electric shock causes the membrane of the mature schizonts to rupture and the merozoites thus released can internalize the linearized plasmids. Fifty microlitres of complete culture medium were immediately added to the content of the cuvette and two 3-week-old Swiss mice were immediately infected with 100 μl, each, of transfected merozoites. On D1 after infection, the selection drug, pyrimethamine, was administered in the drinking water and smears were performed every day. The pyrimethamine solution was prepared in the following way: 70 mg of pyrimethamine (Sigma Aldrich), 10 ml DMSO, qs 1 1 water, pH 3.5-5.5. When the mice became positive for the presence of parasites (approximately D6 after infection) and the parasitaemia reached between 2% and 10%, their blood was taken by intracardiac puncture.

### Parasite Culture Conditions and Inoculation

The C57BL/6 mice were infected, intraperitoneally, with 200 μl, each, of a vial containing 10⁷ pRBCs/200 μl. When the parasitaemia reached between 1% and 5% (5-6 days after infection), approximately 100 μl of blood were taken from the cheek of the animal, into a tube containing heparin. This blood was washed twice with 1 ml of cold PBS and centrifuged for 5 min at 3800 rpm. After dilution to 1/1000 and cell counting, the C57BL/6 mice were infected with 10⁵ pRBCs.

### Parasitaemia Measurement

The parasitaemia is analysed by counting parasitized red blood cells (pRBCs) in stained smears under a microscope.

Preparation of Parasitized Red Blood Cells and Freezing of pRBCs

The C57BL/6 mice are infected, intraperitoneally, with 200 μl, each, of a vial containing 10⁷ pRBCs/200 μl.

Two types of parasite stocks are prepared.

### Stabilates:

The blood is taken from infected mice, by intracardiac puncture (animal anaesthetized), when the parasitaemia reaches between 3% and 10% (5 to 6 days after infection with 10⁷ parasitized red blood cells), into a tube containing heparin and always kept in ice until freezing in liquid nitrogen in the following cryopreserving mixture: 1 vol pure glycerol (Sigma Aldrich)+9 vol of Alsever's Solution (Sigma Aldrich). The stabilates are prepared with 1 vol of the blood taken, to which 2 vol of the cryopreserving mixture are added. The aliquot portions are frozen in liquid nitrogen (approximately 300 μl per mouse) and subsequently stored at −80° C.

### Vials:

As previously, the blood is taken by intracardiac puncture, but when the parasitaemia reaches between 1% and 5%. This blood is washed several times with cold PBS and centrifuged for 5 min at 3800 rpm. Vials containing 10⁷ pRBCs/200 μl are then prepared in a cryopreserving mixture, see previous paragraph, after counting the number of parasitized red blood cells.

### Results

### Role of the HMGB2 Protein in the Development of the PbNK65 Parasite

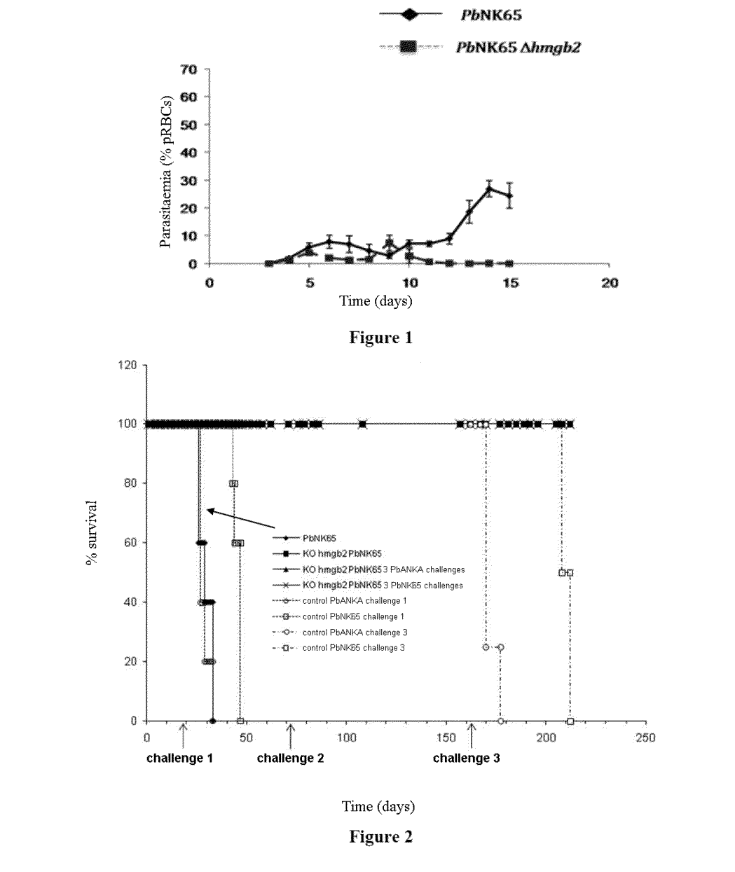

The inventors demonstrated that the inactivation of the hmgb2 gene hindered the development of PbNK65 Δhmgb2 in the C57BL/6 (CM-S) mouse with clearance of the parasite 10 to 15 days after the infection (D10-D15) according to the infecting dose of parasites. FIG. 1 represents the mean of 3 experiments (n=5 mice per experiment) during which the C57BL/6 mice were infected with red blood cells parasitized with wild-type PbNK65 or PbNK65 Δhmgb2. This clearance of the mutated parasite was observed approximately 15 days after infection. The mutated parasite was no longer detected in the blood smears, whereas the development of the wild-type parasite continued until death due to hyperparasitaemia approximately 25 days after infection. This is because, whereas the wild-type parasite continues to develop, the infection with PbNK65 Δhmgb2 induces an immune response in the C57BL/6 mouse which modifies the development of this parasite. Several independent mutated clones were obtained showing the same clearance kinetics during their development in the C57BL/6 mice.

Sterile Protection Induced by Primary Infection With PbNK65 Δhmgb2

Several experiments were carried out with from one to three successive infection challenges in C57BL/6 mice subjected to primary infection with the mutated parasite PbNK65 Δhmgb2 (10⁵ pRBCs intraperitoneally or intravenously). When the parasite 10 was no longer detected in the blood of the C57BL/6 mice (around D19), the inventors performed the first infection challenge (D19), then the second on D71 and the third on D162 with two highly pathogenic wild-type parasites:

1. the wild-type homologous parasite PbNK65 which results in death of the mice due to hyperparasitaemia and

2. the wild-type heterologous parasite PbANKA which induces death of the mice due to cerebral malaria.

FIG. 2 gives the results of an experiment with three successive infection challenges. All the mice subjected to primary infection with PbNK65 Δhmgb2 survived the three infection challenges, with sterile protection being maintained for at least 7 months. At each challenge, the inventors verified that the development of the wild-type PbNK65 parasite in naive mice continues up to approximately 25 days, at which point the mice begin to die from hyperparasitaemia, and that the development of the wild-type PbANKA parasite in naive mice continues up to approximately 7 days, at which time the mice die from cerebral malaria (controls for the pathogenicity of the parasites and the age of the mice).

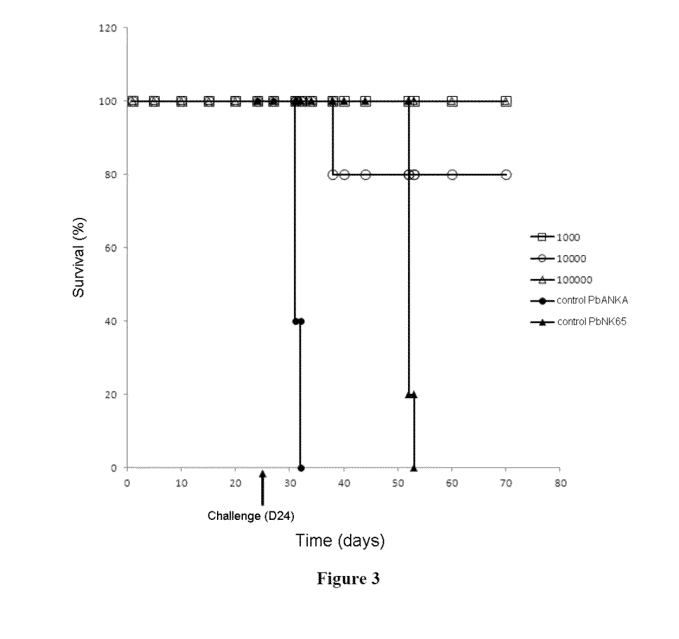

In order to determine the smallest dose of red blood cells infected with PbNK65 Δhmgb2 which is necessary and sufficient to promote sterile protection, infection challenges were carried out on mice subjected to primary infection with 10⁵ to 10³ pRBCs (FIG. 3) and even 10² pRBCs (results not shown). These experiments showed that the injection of 10² or 10³ pRBCs made it possible to induce sterile protection during an infection challenge carried out by intravenous injection of 10⁵ wild-type PbNK65 or PbANKA pRBCs.

### CONCLUSION

These results show that sterile immunity (the parasites are no longer detected in the peripheral blood) can be induced after a primary infection with the PbNK65 Δhmgb2 parasite. This protection extends not only to the wild-type homologous parasite 5 PbNK65 but also to the heterologous parasite PbANKA capable of inducing cerebral malaria.

Patents, patent applications, publications, product descriptions, and protocols which are cited throughout this application are incorporated herein by reference in their entireties for all purposes.

To facilitate an understanding of the principles and features of the invention, various illustrative embodiments are described in this specification. Although exemplary embodiments are explained in detail, it is to be understood that other embodiments are contemplated. Accordingly, neither the invention, nor any of the appended claims, is limited in its scope to specific examples or embodiments herein, or to the details of construction and arrangement of components set forth in the foregoing description or illustrated in the drawings. The invention is capable of other embodiments and of being practiced, carried out, and claimed in various ways.

### LITERATURE REFERENCES

Aly et al. Cell Microbiol. 2010 Jul;12(7):930-8,

Boyle et al. Proc Natl Acad Sci U S A. 2010 Aug 10;107(32):14378-83

Briquet et al. Eukaryotic Cell, 2006, 5: 672-82,

Buffet et al. Proc Natl Acad Sci U S A. 1999 Oct. 26;96(22):12743-8.

de Koning-Ward et al. Annu Rev Microbiol, 2000. 54: 157-85,

De Sa et al. Parasitology Research, 2009, 105: 275-279,

Gissot et al. J Mol Biol. 2005, 346(1):29-42,

Hoffman et al. Human Vaccines 6:1, 97-106, 2010

Kumar et al. Parasitology International, 2008, 57: 150-157,

Leef et al. Bull World Health Organ. 1979;57 Suppl 1:87-91,

Lotze and Tracey, Nature Reviews Immunology, Volume 5 April 2005, 331

McRobert and McConkey. Mol Biochem Parasitol. 2002 Feb.;119(2):273-8,

Mohmmed et al. Biochem Biophys Res Commun. 2003 Sep. 26;309(3):506-11,

Nacer et al., PLoS One. 2011;6(12):e29039.

Orjih et al. Am J Trop Med Hyg. 1980 May;29(3):343-7,

Pombo et al. Lancet. 2002 Aug. 24;360(9333):610-7,

Traoré et al., Am. J. Trop. Med. Hyg, 62(1) 2000, pp 38-44

Triglia et al. Mol. Microbiol. 2000, 38:706-718.

Trenholme et al., PNAS April 11, 2000 vol. 97 no. 8 4029-403

Ting et al. Nat. Med., 2008, 14 (9): 954-958

Thathy and Menard. Methods Mol Med. 2002;72:317-31.

Trager and Jensen. Science 193:673-5, 1976.

Udeinya et al. Exp Parasitol. 1983 Oct.;56(2):207-14.

1. A method of preventing malaria or cerebral malaria in a mammal, comprising administering a live parasite belonging to the Plasmodium genus in which the function of the hmgb2 gene is inactivated.

2. The method of claim 1, wherein the strain of the parasite is selected from the group consisting of Plasmodium berghei, Plasmodium falciparum, Plasmodium vivax, Plasmodium ovale, Plasmodium malariae and Plasmodium knowlesi.

3. The method of claim 2, wherein the strain of the parasite is selected from the group consisting of Plasmodium falciparum, Plasmodium vivax, Plasmodium ovale and Plasmodium malariae.

4. The method of claim 3, wherein the strain of the parasite is Plasmodium falciparum.

5. The method of claim 4, wherein the parasite in its wild-type form does not cause cerebral malaria.

6. The method of claim 1, wherein the strain of the parasite is Plasmodium berghei NK65.

7. The method of claim 1, wherein the strain of the parasite is a Plasmodium falciparum strain that is non-cytoadherent or that has a reduced cytoadherence capacity.

8. The method of claim 7, wherein the strain of the parasite is a Plasmodium falciparum strain that has a reduced cytoadherence capacity.

9. The method of claim 1, wherein the parasite is in the form of non-intra-erythrocytic merozoites.

10. The method of claim 1, wherein the parasite is in an intra-erythrocytic form.

11. The method of claim 10, wherein the parasite is in the form of intra-erythrocytic trophozoites, merozoites or schizonts.

12. The method of claim 11, wherein the parasite is in the form of intra-erythrocytic merozoites or schizonts.

13. The method of claim 1, wherein the parasite is in the form of sporozoites.

14. The method of claim 1, wherein the function of the hmgb2 gene is inactivated by total or partial deletion of said gene.

15. The method of claim 14, wherein the function of the hmgb2 gene is inactivated by total deletion of said gene.

16. The method of claim 15, wherein the coding region of the hmgb2 gene is replaced with a selectable marker.

17. The method of claim 16, wherein the selectable marker is the human gene encoding dihydrofolate reductase.

18. The method of claim 1, wherein the function of the hmgb2 gene is inactivated by means of an interfering RNA which blocks or decreases the translation of the HMGB2 protein.

19. The method of claim 1, wherein the function of one or more other genes is inactivated.

20. The method of claim 19, wherein the other gene of which the function is inactivated is selected from the group consisting of the genes encoding purine nucleoside phosphorylase, nucleoside transporter 1, UIS3, UIS4, p52 and p36, and a combination thereof.

21. An immunogenic composition comprising an immunologically effective amount of live parasites belonging to one or more strains of the Plasmodium genus in which the function of the hmgb2 gene is inactivated, and one or more pharmaceutically acceptable excipients or supports.

22. A malaria vaccine comprising a live parasite belonging to the Plasmodium genus in which the function of the hmgb2 gene is inactivated and one or more pharmaceutically acceptable excipients or supports.

23. The immunogenic composition according to claim 21, further comprising one or more immunological adjuvants.

24. The immunogenic composition according to claim 23, wherein the immunological adjuvant is selected from the group consisting of adjuvants of muramyl peptide type; trehalose dimycolate (TDM); lipopolysaccharide (LPS); monophosphoryl lipid A (MPL); carboxymethylcellulose; complete Freund's adjuvant; incomplete Freund's adjuvant; adjuvants of “oil-in-water” emulsion type optionally supplemented with squalene or squalane; mineral adjuvants; bacterial toxins; CpG oligodeoxynucleotides; saponins; synthetic copolymers; cytokines and imidazoquinolones, and a combination thereof.

25. The immunogenic composition according to claim 21, formulated so as to be administered parenterally.

26. The immunogenic composition according to claim 21, wherein the strains of Plasmodium parasite are selected from the group consisting of Plasmodium berghei, Plasmodium falciparum, Plasmodium vivax, Plasmodium ovale, Plasmodium malariae, and Plasmodium knowlesi, in which the function of the hmgb2 gene is inactivated.

27. The immunogenic composition according to claim 21 comprising between 10 10⁶ of said parasites in an intra-erythrocytic form per dose unit.

28. The immunogenic composition according to claim 21, comprising between 10³ and 10⁷ parasites in the form of sporozoites per dose unit.

29. The immunogenic composition according to claim 21, comprising between 10² and 10⁷ parasites in the form of non-intra-erythrocytic merozoites per dose unit.

30. (canceled)

31. A method for immunizing a subject against malaria, comprising the administration of an immunogenic composition according to claim 21 to said subject.

32. The method according to claim 31, wherein the composition is administered parenterally.

33. The method according to claim 31, wherein the composition is administered subcutaneously, intramuscularly, intradermally or intravenously.


SEQUENCE LISTING (14 sequences)

SEQ ID NO: 1 - DNA, 300 bp, Plasmodium falciparum 3D7
atggcttcaaaatctcaaaagaaagtattaaaaaaacaaaacaaaaaaaagaaaaaagacccattagctccaaagagagctttgtctgcctatatgttttatgttaaggataagagactagaaataataaaagaaaaaccagaattagcaaaagatgttgcacaagttggtaaattgataggtgaagcttggggacaattaagcccagctcaaaaagcaccatatgaaaagaaagcccaattagacaaagtacgatattcaaaagaaatagaagaatatagaaagaaaaatcaagaataa

SEQ ID NO: 2 - protein, 99 aa, Plasmodium falciparum 3D7
MASKSQKKVLKKQNKKKKKDPLAPKRALSAYMFYVKDKRLEIIKEKPELAKDVAQVGKLIGEAWGQLSPAQKAPYEKKAQLDKVRYSKEIEEYRKKNQE

SEQ ID NO: 3 - DNA, 1275 bp, Plasmodium vivax Sal-1
ttggcgttaaagctaacgttaaagcgaacgttaaagttaagttgaatttgcctcccccacccccctgtgtgtttttttttttttttttttcccaatccgtacaggtatatggttacgcttgtgcacgcatacacttcctccccccacgagcatttcaattttttttaaaaattaaactgcatagtacagttaatgacccctcgtgtgaatattaaaaggaaatttccttcccatccctatcattttgtttcccttttattttcccctttcttcctccctttattttttcccctttttttccattttccacacaacttccacattttaaatcctcacttggaacttccgaacgggaaaaaacaaacacaggaattagcaactgtaacgacgaagcagatgagtaacaaaatggccgccaaatctcaaaagaaagtcttaaaaaagcaaaataaaaggaaaaagaaggaccccttagctccaaagagagccttgtcagcctacatgttctatgtcaaggacaagaggctcgaattaatcaaagagagaccagagctcgccaaagatgttgcgcaagtgggcaagttggttggagaggcctgggggcagttaagcgccgcccagaagactccgtacgaaaagaaggcgcagttggacaaagtcaggtactccaaggagatagaggaatacaggaagacaaaaaaggagtagaagccgcctgccgggggcgccttacgtaatcatccacgcgaaaactcatccacacatgtacccacccacacgcacatgcacctgtttttaccccggcgcatacgtatgcaaaacgcgtacgcatgcccctgcacgaggggaaattctttttgcaggcagcgcgtgcatgcgaagggacaactcttttttttatcattcctgctacgttgcaaggaggcagcgctgcagtgtacttttcgcgtgtgcctctatgtgcatatgtacgggcatatgtatgtattatatatctgcgcgcatgtagcagctcccctgagaaccgcccaatgcacacatacacacacgcgaacagctcacgggctggtatgtttgtaaccccctcacgtacgttcacttgtgcgcgtgcctcactgcatatgtttacgctcgtagtccacgtatgtgtacacacatatgcatgactatactatatatgtcgactgctgcgtttagcaatttttttttttttcataactcactattgtagaaatattcgcaactctctttttttttttttctcttacgaagaat

SEQ ID NO: 4 - protein, 171 aa, Plasmodium vivax Sal-1
MTPRVNIKRKFPSHPYHFVSLLFSPFFLPLFFPLFFHFPHNFHILNPHLELPNGKKQTQELATVTTKQMSNKMAAKSQKKVLKKQNKRKKKDPLAPKRALSAYMFYVKDKRLELIKERPELAKDVAQVGKLVGEAWGQLSAAQKTPYEKKAQLDKVRYSKEIEEYRKTKKE

SEQ ID NO: 5 - DNA, 297 bp, Plasmodium berghei str. ANKA
atggcaactaaaacacaaaagaaagttataaaaaagcaaaacaaaaaaaagaaaaaagatccattagctccaaagagagcattatctgcttatatgttttatgtcaaagacaaaagattagaaataattcaagaaaggccagagctagctaaagaagttgcacaagttggaaaactaattggagaagcttggggacagttaactccagctcaaaaagctccatatgaaaaaaaagcagaattagataaagttcgttattcaaaagaaatagaagaatatagaaaaaccaaggaataa

SEQ ID NO: 6 - protein, 98 aa, Plasmodium berghei str. ANKA
MATKTQKKVIKKQNKKKKKDPLAPKRALSAYMFYVKDKRLEIIQERPELAKEVAQVGKLIGEAWGQLTPAQKAPYEKKAELDKVRYSKEIEEYRKTKE

SEQ ID NO: 7 - DNA, 300 bp, Plasmodium knowlesi
atggcagcaaagtctcaaaagaaagttttaaggaagcaaaacaaacggaagaagaaggaccccttagctccaaagagagccctgtcagcctacatgttctatgtaaaggacaagcgactcgaattaatcaaagagaaaccagagctcgccagaaatgttgcacaagtaggcaagttggttggtgaggcctgggggaagttaagtgccgcgcagaagactccgtacgaaaaaaaggcgcagttagacaaagtcaggtactccaaggagatagaagaatacaggaagacaaaaaatgagtag

SEQ ID NO: 8 - protein, 99 aa, Plasmodium knowlesi
MAAKSQKKVLRKQNKRKKKDPLAPKRALSAYMFYVKDKRLELIKEKPELARNVAQVGKLVGEAWGKLSAAQKTPYEKKAQLDKVRYSKEIEEYRKTKNE

SEQ ID NO: 9 - DNA, 381 bp, Plasmodium yoelii
atgtacatatattatatcctatcaagtgtgttgtatatgtcttcttacatattctatgagtttgtttcaaaacagatagacaaaatggcaactaaaacacaaaagaaggttataaaaaagcaaaacaaaaaaaagaaaaaagacccattagctccaaagagagcattgtctgcttatatgttttatgtaaaagacaaaagattggaaataattcaagaaagaccagaactagctaaagaagttgcacaagttggaaaattaatcggagaggcatggggacaattaaccccagctcaaaaagccccatatgaaaaaaaagctgaattagataaagttcgttattccaaagaaatagaagaatatagaaaaaccaaagaataa

SEQ ID NO: 10 - protein, 126 aa, Plasmodium yoelii
MYIYYILSSVLYMSSYIFYEFVSKQIDKMATKTQKKVIKKQNKKKKKDPLAPKRALSAYMFYVKDKRLEIIQERPELAKEVAQVGKLIGEAWGQLTPAQKAPYEKKAELDKVRYSKEIEEYRKTKE

SEQ ID NO: 11 - DNA, 40 bp, Artificial
  sense HR1 PCR primer
cgatgcgggcccaaaaaggtaaatatgaaaaagaaaggtt

SEQ ID NO: 12 - DNA, 35 bp, Artificial
  antisense HR1 PCR primer
cgatgccccgggtttgcaatggaaacatatcagtt

SEQ ID NO: 13 - DNA, 41 bp, Artificial
  sense HR2 PCR primer
ccagtgagtgcggccgcggagccttttgtatgtgctttttg

SEQ ID NO: 14 - DNA, 40 bp, Artificial
  antisense HR2 PCR primer
agctggcgcgccaagtattgcagtagcattttctttaaat